(19) United States
(12) Plant Patent Application Publication
(10) Pub. No.: US 2019/0183005 P1
(43) Pub. Date: Jun. 13, 2019

(54) Cannabis plant named ' PRIMO CHERRY'

(50) ;

(71) Applicant: Biotech Institute, LLC, Westlake Village, CA (US)

(72) Inventors: Mark Anthony Lewis, Westlake Village, CA (US); Steven Robert Haba, Westlake Village, CA (US)

(21) Appl. No.: 15/999,233

(22) Filed: Aug. 28, 2018

Related U.S. Application Data
(60) Provisional application No. 62/596,561, filed on Dec. 8, 2017.

(51) Int. Cl. A01H 6/28

(52) U.S. Cl. PLT/263.1

(57) ABSTRACT

The present invention provides a new and distinct cannabis cultivar designated as ‘PRIMO CHERRY’.

### CROSS-REFERENCE TO RELATED APPLICATIONS

This application claims priority to, and the benefit of U.S. Provisional Patent Application Ser. No. 62/596,561, filed Dec. 8, 2017, which is hereby incorporated by reference in its entirety for all purposes.

Latin name of genus and species: Cannabis hybrid (mixed background).

Variety denomination: ‘PRIMO CHERRY’.

### BACKGROUND OF THE INVENTION

The present invention relates to a new and distinct cannabis cultivar designated as ‘PRIMO CHERRY’.

This new cultivar is the result of controlled-crosses between proprietary cultivars made by the inventors. The new cultivar of ‘PRIMO CHERRY’ was asexually reproduced via a ‘cutting’ and ‘cloning’ method by the inventors at Salinas, Calif. Asexual clones from the original source have been tested in greenhouses, nurseries, and/or fields. The properties of each cultivar were found to be transmissible by such asexual reproduction. The cultivar is stable and reproduces true to type in successive generations of asexual reproduction.

### TAXONOMY AND NOMENCLATURE

Cannabis, more commonly known as marijuana, is a genus of flowering plants that includes at least three species, Cannabis sativa, Cannabis indica, and Cannabis ruderalis as determined by plant phenotypes and secondary metabolite profiles. In practice however, cannabis nomenclature is often used incorrectly or interchangeably. Cannabis literature can be found referring to all cannabis varieties as “sativas” or all cannabinoid producing plants as “indicas”. Indeed the promiscuous crosses of indoor cannabis breeding programs have made it difficult to distinguish varieties, with most cannabis being sold in the United States having features of both sativa and indica species.

Human cultivation history of Cannabis dates back 8000 years (Schultes, R E., 1970, Random thoughts and queries on the botany of Cannabis. Pages 11-38 in: C R B Joyce, and S H Curry eds., THE BOTANY AND CHEMISTRY OF CANNABIS. J. & A. Churchill. London, England). Hemp cloth recovered in Europe dates back 6000 years (Small, E, Beckstead, H D, and Chan, A, 1975, The evolution of cannabinoid phenotypes in Cannabis, ECONOMIC BOTANY 29(3):219-232). The written record of the pharmacologic properties of Cannabis goes back more than 4000 years (Ti, H. 2737 BC: NEI JING SU WEN HUANG TI, Yellow Emperor's Classic on Internal Medicine; referred to without citation in Small et al. 1975 Supra).

The taxonomy and nomenclature of the highly variable genus Cannabis (Emboden, W A, 1974, ECONOMIC BOTANY 28(3):304-310; Small, E and Cronquist, A, 1976, TAXON 25(4):405-435; Small E and Cronquist, A, 1977, TAXON 26(1): 110; Hillig, K W and Mahlberg, P G, 2004, American Journal of Botany 91(6):966-975), remains in question. This is in spite of the fact that its formal scientific name, ‘Cannabis sativa L.’, assigned by Carolus Linneaus (Linnaeus, C, 1753, SPECIES PLANTARUM, 2:1027, Salvius, Stockholm, Facsimile edition, 1957-1959, Ray Society, London, U.K.), is one of the oldest established names in botanical history and is still accepted to this day. Another species in the genus, ‘Cannabis indica Lam.’ was formally named somewhat later (de Lamarck, J B, 1785, ENCYCLOPEDIE METHODIQUE DE BOTANIQUE, 1(2):694-695), but is still very old in botanical history. In 1785, Jean-Baptiste Lamarck published a description of a second species of Cannabis, which he named Cannabis indica. Lamarck based his description of the newly named species on plant specimens collected in India. C. indica was described as relatively short, conical, and densely branched, whereas C. sativa was described as tall and laxly branched (Schultes R. E. et al, 1974, Harvard University Botanical Museum Leaflets, 23: 337-367). C. indica plants were also described as having short, broad leaflets whereas those of C. sativa were characterized as relatively long and narrow (Anderson L. C., 1980, Harvard University Botanical Museum Leaflets, 28: 61-69). C. indica plants conforming to Schultes' and Anderson's descriptions may have originated from the Hindu Kush mountain range. Because of the often harsh and variable (extremely cold winters, and warm summers) climate of those parts, C. indica is well-suited for cultivation in temperate climates.

Three other species names were proposed in the 1800s to distinguish plants with presumably different characteristics (C. macrosperma Stokes, C. chinensis Delile, C. gigantean Vilmorin), none of which are accepted today, although the epithet “indica” lives on as a subspecies of C. sativa (‘C. sativa ssp. indica Lam.’, Small and Cronquist 1976 Supra).

In the 20th century, two new names were added to the liturgy of proposed ‘Cannabis species: C. ruderalis’ Janischevsky and a hybrid, x ‘C. intersita’ Sojak. (Small, E, Jui, P Y, and Lefkovitch, L P, 1976, SYSTEMATIC BOTANY 1(1): 67-84; Small and Cronquist 1976 Supra). Further, numerous names have been proposed for horticultural variants of ‘Cannabis’ but as of 1976, “very few of these have been validly published as formal taxa under the International Code of Botanical Nomenclature” (Small and Cronquist 1976 Supra). Moreover, other recent work continues to focus on higher-order evolutionary relationships of the genus. Cannabis has been variously ascribed as belonging to mulberry family (Moraceae) (Engler, H G A, Ulmaceae, Moraceae and Urticaceae, pages 59-118 in: A. Engler and K. Prantl eds., 1889, DIE NATURLICHEN PFLANZENFAMILIEN 3(1). W. Engelmann, Leipzig, Germany; Judd, W S, Sanders, R W, and Donoghue, M J, 1994, HARVARD PAPERS IN BOTANY 5: 1-51; Humphries, C J and Blackmore, S, A review of the classification of the Moraceae, pages 267-277 In: Crane and Blackmore 1989 id.); nettle family (Urticaceae) (Berg, C C, Systematics and phylogeny of the Urticales, pages 193-220, in: P. R. Crane and S. Blackmore eds., 1989, EVOLUTION, SYSTEMATIC, AND FOSSIL HISTORY OF THE HAMAMELIDAE, VOL. 2, HIGHER HAMAMELIDAE, Clarendon Press, Oxford, U.K.); and most recently in its own family with hops (Humulus), Cannabaceae, or hemp family (Sytsma, K J, et al, 2002, AMERICAN JOURNAL OF BOTANY 89(9):1531-1546). While the work of Small and Cronquist 1976 Supra, seemed to effectively confine the genus to a single species with 2 subspecies (C. sativa s., C. s. indica), each with two varieties (C. s. s. var. sativa, C. s. s. var. spontanea; C. s. i. var. indica, C. s. i. var. Kafiristanica) largely on the basis of chemotaxonomy and interfertility of all forms, more recent work (Sytsma et al. 2002 Supra), proposes a two-species concept, resurrecting the binomial C. indica Lam. Since Sytsma et al. (2002) provides no key for discriminating between the species, the dichotomous key of Small and Cronquist (1976), which accounts for all forms in nature, whether wild or domesticated, is preferred to classify the characteristics of the plants.

### BRIEF SUMMARY OF THE INVENTION

This invention relates to a new and distinctive cannabis cultivar designated as ‘PRIMO CHERRY’.

The objective of the breeding program which produced novel plants disclosed herein was primarily to develop a cannabis cultivar with its unique blend of various cannabinoids and/or terpenes for (a) medicinal effects such as improving appetite and reducing nausea, vomiting and/or chronic pain, as well as neurological and cardiovascular effects, (b) psychoactive effects such as increased motivation and energetic behavior rather than indifference, passiveness and lethargy, and (c) recreational effects with enhanced enjoyment such as food and aroma.

As used herein, the term “cultivar” is used interchangeably with “variety”, “strain”, and/or “clone”.

Cannabis plants produce a unique family of terpeno-phenolic compounds. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. The cannabis plant has at least 545 distinct compounds that span 20 chemical classes including cannabinoids, terpenes, terpenoids, amino acids, nitrogenous compounds, simple alcohols, aldehydes, ketones, esters, lactones, acids, fatty acids, steroids, non-cannabinoid phenols, pigments, flavonoids, vitamins, proteins, enzymes, glycoproteins, and hydrocarbons. Terpenes and/or cannabinoids, in particular, have shown great potential in terms of medicinal value.

Terpenes and/or cannabinoids have been shown to be largely responsible for beneficial effects of a cannabis plant. In fact, each cannabis plant has the varying concentrations of medically viable compounds depending on different strains (genotypes) and their resulting chemotypes. Even a small variation in terpene and/or cannabinoid concentration can cause noticeable differences in the entourage and/or synergistic effects of a cannabis plant, which distinguishes one variety from another. Research shows that it relies heavily on the physiological effects produced by terpenes and/or cannabinoids.

Over 100 different kinds of terpenes have been identified in cannabis plants although not being as well-studied as cannabinoids, they are instrumental in giving rise to the physiological and psychoactive effects in cannabis.

Terpenes are a large and diverse class of organic compounds, produced by a variety of plants. They are often strong smelling and thus may have had a protective function. Terpenes are an important component, not only influencing taste and smell of each cannabis strain but also influencing its effects on the mind and body of a subject such as humans and animals. Terpenes are a classification of organic molecules that are found in a wide variety of plants and animals. These molecules are known for their characteristic scents and flavors. The varying terpene concentrations found in cannabis plants directly influence the resulting taste and smell, as well as the observed effects. Non-limiting examples of terpenes include Hemiterpenes, Monoterpenes, Sesquiterpenes, Diterpenes, Sesterterpenes, Triterpenes, Sesquarterpenes, Tetraterpenes, Polyterpenes, and Norisoprenoids. The main terpenes found in cannabis plants include, but are not limited to, myrcene, limonene, caryophyllene, pinene, terpinene, terpinolene, camphene, terpineol, phellandrene, carene, humulene, pulegone, sabinene, geraniol, linalool, fenchol, borneol, eucalyptol, and nerolidol.

Cannabinoids are the most studied group of the main physiologically active secondary metabolites in cannabis. The classical cannabinoids are concentrated in a viscous resin produced in structures known as glandular trichomes. At least 113 different cannabinoids have been isolated from cannabis plants. The main classes of cannabinoids from cannabis include tetrahydrocannabinol (THC), cannabidiol (CBD), cannabigerol (CBG), and cannabinol (CBN). Cannabinoid can be at least one of a group comprising tetrahydrocannabinol (THC), cannabidiol (CBD), cannabigerol (CBG), cannabinol (CBN) cannabichromene (CBC), cannabinodiol (CBDL), cannabicyclol (CBL), cannabivarin (CBV), tetrahydrocannabivarin (THCV), cannabidivarin (CBDV), cannabigerovarin (CBGV), cannabichromevarin (CBCV), cannabigerol monomethyl ether (CBGM), cannabielsoin (CBE), cannabicitran (CBT), cannabinol propyl variant (CBNV), cannabitriol (CBO), tetrahydrocannabinolic acid (THCA), tetrahydrocannabivarinic acid (THCVA), cannabidiolic acid (CBDA), cannabigerolic acid (CBGA) and cannabinerolic acid.

Most cannabinoids exist in two forms, as acids and in neutral (decarboxylated) forms. The acid form is designated by an “A” at the end of its acronym (i.e. THCA). The cannabinoids in their acid forms (those ending in “-A”) can be converted to their non-acid forms through a process called decarboxylation when the sample is heated. The phytocannabinoids are synthesized in the plant as acid forms. While some decarboxylation does occur in the plant, it increases significantly post-harvest and the kinetics increase at high temperatures (Flores-Sanchez and Verpoorte, 2008, Plant Cell Physiol. 49(12): 1767-1782). The biologically active forms for human consumption are the neutral forms. Decarboxylation is usually achieved by thorough drying of the plant material followed by heating it, often by combustion, vaporization, heating, or baking in an oven. Unless otherwise noted, references to cannabinoids in a plant include both the acidic and decarboxylated versions (e.g., CBD and CBDA).

The molecules lose mass through the process of decarboxylation. In order to find the total theoretical active cannabinoids, the acid forms should be multiplied by 87.7%. For example, THCA can be converted to active THC using the formula: THCA×0.877=THC. The maximum THC for the sample is: THCₘₐₓ=(THCA×0.877)+THC. This method has been validated according to the principles of the International Conference on Harmonization. Similarly, CBDA can be converted to active CBD and the yield is determined using the yield formula: CBDA×0.877=CBD. Also the maximum amount of CBD yielded, i.e. max CBD for the sample is: CBDₘₐₓ=(CBDA×0.877)+CBD. Additionally, CBGA can be converted to active CBG by multiplying 87.8% to CBGA. Thus, the maximum amount of CBG is: CBGₘₐₓ=(CBGA×0.878)+CBG.

The biologically active chemicals found in plants, phytochemicals, affect the normal structure or function of the human body and in some cases treat disease. The mechanisms for the medicinal and psychoactive properties of a cannabis plant, like any medicinal herb, produce the pharmacologic effects of its phytochemicals, and the key phytochemicals for a medical cannabis plant are cannabinoids and terpenes.

Δ9-Tetrahydrocannabinol (THC) is a psychoactive cannabinoid responsible for many of the effects such as mild to moderate pain relief, relaxation, insomnia and appetite stimulation. THC has been demonstrated to have anti-depressant effects. The majority of strains range from 12-21% THC with very potent and carefully prepared strains reaching even higher. While Δ9-Tetrahydrocannabinol (THC) is also implicated in the treatment of disease, the psychotropic activity of THC makes it undesirable for some patients and/or indications.

Tetrahydrocannabinol, THC, is the primary psychoactive and medicinal cannabinoid and is the result of the decarboxylation of tetrahydrocannabinolic acid (THCA), its acidic precursor. THCA, (6ar,10ar)-1-hydroxy-6,6,9-trimethyl-3 -pentyl-6a,7,8,10a-tetrahydro-6h-benzochromene-2-carboxylic acid, is found in the trichomes of the plant and converted into THC, which actually exists in only minute quantities in the living plant, after harvest and drying.

Cannabidiol (CBD) is one of the principal cannabinoids found in a cannabis plant and is largely considered to the most medically significant. CBD occurs in many strains, at low levels, <1%. In some cases, CBD can be the dominant cannabinoid, as high as 15% by weight. CBD is non-psychoactive, meaning that unlike THC, CBD does not cause a noticeable “high”. CBD has been implicated for medical properties in the treatment of a wide variety of diseases and symptoms, including cancer, nausea, chronic pain, spasms, seizures/epilepsy, anxiety, psoriasis, Crohn's disease, rheumatoid arthritis, diabetes, schizophrenia, post-traumatic stress disorder (PTSD), alcoholism, strokes, multiple sclerosis, and cardiovascular disease. CBD also has been reported to act as a muscle relaxant, antibiotic, anti-inflammatory, and bone stimulant, as well as to improve blood circulation, cause drowsiness, and protect the nervous system. It can provide relief for chronic pain due to muscle spasticity, convulsions and inflammation, as well as effective relief from anxiety-related disorders. It can offer relief for patients with Multiple Sclerosis (MS), Fibromyalgia and Epilepsy. CBD has also been shown to inhibit cancer cell growth when injected into breast and brain tumors in combination with THC.

A cannabis cultivar can be used to achieve the desire of patients to be treated with CBD without the adverse side-effects (e.g., psychoactivity) of THC.

Cannabichromene (CBC) is a rare, non-psychoactive cannabinoid, usually found at low levels (<1%) when present. It has been shown to have anti-depressant effects and to improve the pain-relieving effects of THC. Studies have demonstrated that CBC has sedative effects such as promoting relaxation.

Cannabidiol (CBD) and cannabichromene (CBC) are both non-psychoactive and end products of CBG metabolism, like THC, so that they can be used medically.

Cannabigerol (CBG) is a non-psychoactive cannabinoid. CBG-acid is the precursor to both THC-acid and CBD-acid in the plant usually found at low levels (<1%) when present. It has been demonstrated to have both pain relieving and inflammation reducing effects. CBG reduces intraocular pressure, associated with glaucoma. CBG has been shown to have antibiotic properties and to inhibit platelet aggregation, which slows the rate of blood clotting. While Cannabigerol (CBG), is not considered psychoactive, it is known to block the psychoactive effects of THC and is considered medically active in a variety of conditions. Its precursor, cannabigerolic acid, CBGA, (E)-3-(3,7-Dimethyl-2,6-octadienyl)-2,4-dihydroxy-6-pentylbenzoic acid, is being studied medically.

Cannabinol (CBN) is an oxidative degradation product of THC. It may result from improper storage or curing and extensive processing, such as when making concentrates. It is usually formed when THC is exposed to UV light and oxygen over time. CBN has some psychoactive properties, less strength than THC. CBN is thought to enhance the dizziness and disorientation that users of cannabis may experience. It may cause feelings of grogginess, but has been shown to reduce heart rate.

High potency cannabis plants contain large quantities of specific terpenes as well as various assortments of others. For instance, a cannabis plant may have a profile with either a high level of, a moderate amount of or a small amount of various terpenes depending on its cultivar and environmental conditions.

Various cultivars of ‘Cannabis’ species have been cultivated in an effort to create a cultivar best suited to meet the interest of inventors according to its own need. The particular plant disclosed herein was discovered in the area where the inventors were intentionally cross-pollinating and cultivating plants described below using standard Mendelian breeding procedures well known to those of ordinary skill in the art. This resulted in the progenies of the inventors' crosses.

The progenies resulting from any selection stage of either the crossing, selfing or backcrossing versions of the breeding regimes of the present invention were asexually reproduced to fix and maintain the desirable THC content, CBs content, terpenes content, the aroma and flavor(s) typical of the desired class, and the other desirable phenotypic and/or genotypic characteristics. The resultant selected cannabis cultivar is designated as ‘PRIMO CHERRY’ disclosed herein.

The inventors reproduced progenies asexually by cutting and cloning. This is the origin of this remarkable new cultivar. The plant has been and continues to be asexually reproduced by cutting and cloning at the inventors' greenhouses, nurseries and/or fields in Salinas, Calif., Oakland, Calif., and/or Washington, D.C.

The following are the most outstanding and distinguishing chemical characteristics of this new cultivar when grown under normal conditions in Salinas, Calif. Chemical analyses of the new cannabis variety and the check variety (or the parental varieties) disclosed herein were performed using standard chemical separation techniques well known to those skilled in the art. Samples for assaying were obtained from flower tissues of the cannabis plant disclosed herein. Cannabinoid composition of this cultivar can be determined by assaying the concentration of at least one cannabinoid in a subset (e.g., sample) of the harvested product.

Table 1 includes detailed information of the cannabis plant named ‘PRIMO CHERRY’ for concentration ranges of terpenes and cannabinoids as tested on flowers at least six different times. The cannabis plant has been tested in a laboratory setting and/or facility to determine cannabinoids and terpenes concentrations in the cannabis plant named ‘PRIMO CHERRY’ according to the procedures provided in Giese et al. (Journal of AOAC International (2015) 98(6):1503-1522).

1) The main terpenes found in ‘PRIMO CHERRY’ are myrcene, alpha-pinene, hexyl butyrate, beta-pinene, limonene, and linalool; and

2) The estimated concentration of the total THCₘₐₓ, CBDₘₐₓ, and CBGₘₐₓ is about 12.38-15.24%, about 5.40-7.13%, and about 0.22-0.33%, respectively, at the time of assaying metabolites from flower samples of ‘PRIMO CHERRY’.

Terpene and cannabinoid profiles of ‘PRIMO CHERRY’ demonstrate that ‘PRIMO CHERRY’ has a phenotypically unique profile, particular insofar as to the level of terpenes and cannabinoids. This data is presented in a tabular form in Table 1.

TABLE 1
Ranges of active Cannabinoids (% by weight)

| | | | | | |
|---|---|---|---|---|---|
| MAX THC | 12.38- | Max CBD | 5.40- | Max CBG | 0.22- |
| | 15.24% | | 7.13% | | 0.33% |

Ranges of Terpenes (% by weight)

| | | | | | |
|---|---|---|---|---|---|
| thujene | 0.00% | gamma- | 0.00% | hexyl hexanoate | 0.02- |
| | | terpinene | | | 0.06% |
| alpha-pinene | 0.37- | linalool oxide | 0.00% | octyl butyrate | 0.00% |
| | 0.73% | | | | |
| camphene | 0.01- | terpinolene | 0.00% | beta-caryophyllene | 0.04- |
| | 0.02% | | | | 0.05% |
| sabinene | 0.00% | fenchone | 0.00% | alpha-humulene | 0.02- |
| | | | | | 0.06% |
| beta-pinene | 0.15- | linalool | 0.05- | cis-nerolidol | 0.00- |
| | 0.33% | | 0.11% | | 0.02% |
| myrcene | 0.19- | fenchol | 0.02- | trans-nerolidol | 0.02% |
| | 0.98% | | 0.04% | | |
| alpha- | 0.00% | — | — | caryophyllene oxide | 0.00- |
| phellandrene | | | | | 0.01% |
| carene | 0.00% | camphor | 0.00% | alpha-bisabolol | 0.01- |
| | | | | | 0.04% |
| alpha- | 0.00% | isoborneol | 0.00% | nerol | 0.00% |
| terpinene | | | | | |
| limonene | 0.10- | (-) borneol | 0.00- | geraniol | 0.00% |
| | 0.32% | | 0.02% | geraniol 0.00% | |
| beta- | 0.00% | menthol | 0.00% | geranyl-acetate | 0.00% |
| phellandrene | | | | | |
| cineole | 0.00% | hexyl butyrate | 0.09- | methyl-eugenol | 0.00% |
| | | | 0.41% | | |
| cis-ocimene | 0.00% | alpha-terpineol | 0.03- | Total Terpenes | 1.28- |
| | | | 0.05% | | 3.22% |
| trans-oceimene | 0.00% | citronellol | 0.00% | — | — |

The cannabis plant named ‘PRIMO CHERRY’ has a complement of terpenes, including but not limited to, relatively high levels of myrcene, alpha-pinene, hexyl butyrate, beta-pinene, limonene, and linalool compared to other terpene compounds. This unique combination of differently concentrated terpenes further distinguishes ‘PRIMO CHERRY’ from other varieties in its odor, its medical qualities, and its effects on mood and mentation.

Physically, there are indications that its use may prevent some cancers and may prevent and/or treat diseases. Indications are that ‘PRIMO CHERRY’ has many medical qualities that make it an important tool to maintain health and deal with illness.

### Asexual reproduction

Asexual reproduction, also known as “cloning”, is a process well known to those of ordinary skill in the art of cannabis production and breeding and includes the following steps.

The cannabis cultivar disclosed herein is asexually propagated via taking cuttings of shoots and putting them in rock wool cubes. These cubes are presoaked with pH adjusted water and kept warm (˜80° C.). Full trays are covered, left under 18 hours of light and allowed to root (7-14 days). Upon root onset, the plantlets are transplanted into rigid 1 gallon containers filled with a proprietary soil mix A and remain in 18 hours of daylight for another 14-21 days. Once root-bound, plants are transplanted into rigid 3 gallon containers filled with proprietary soil mix B. Immediately, the light cycle is altered to 12/12 and flower initiating begins. The plants remain in 12/12 lighting until harvesting. They undergo a propriety nutrient regimen and grow as undisturbed as possible for 60-70 days depending on chemotype analysis.

All sun leaves are removed and the plant is dismantled to result in approximately 12″ branches covered in inflorescences and trichomes. The goal in harvesting is to actually harvest trichome heads but not ‘buds’. Thus, great care is taken not to disturb the trichome heads and as much of the plant remains intact as possible to promote even and slow drying. Slow drying is followed by a one to two months curing process.

Observation of the all female progenies of the original plant has demonstrated that this new and distinct cultivar has fulfilled the objectives and that its distinctive characteristics are firmly fixed and hold true from generation to generation vegetatively propagated from the original plant.

Under careful observation, the unique characteristics of the new cultivar have been uniform, stable and reproduced true to type in successive generations of asexual reproduction.

### DESCRIPTION OF THE DRAWINGS

The accompanying color photographs depict characteristics of the new ‘PRIMO CHERRY’ plants as nearly true as possible to make color reproductions. The overall appearance of the ‘PRIMO CHERRY’ plants in photographs is shown in colors that may differ slightly from the color values described in the detailed botanical description.

FIG. 1 shows an overall view of the ‘PRIMO CHERRY’ plant from the side.

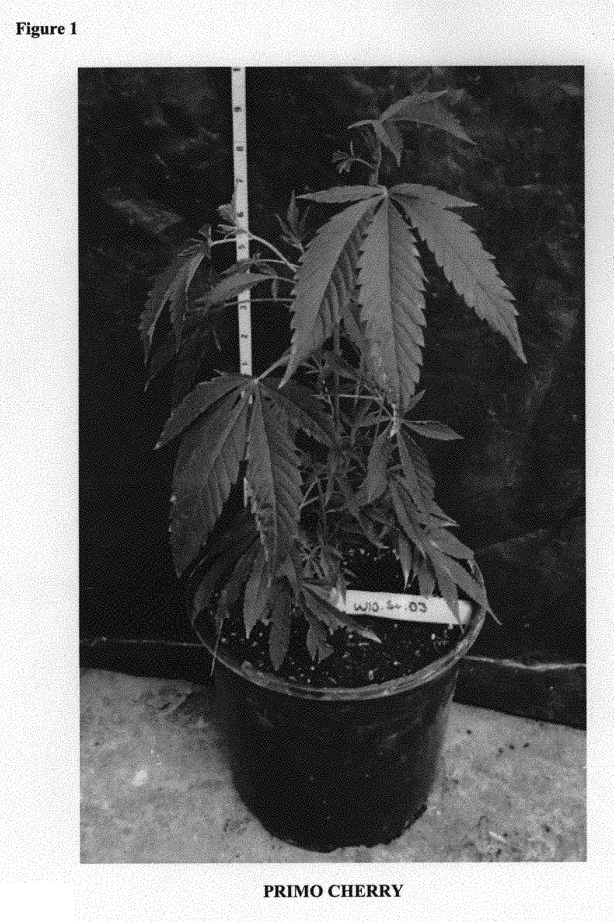

FIG. 2A shows a close view of a single leaf of the check variety BLK03 plant.

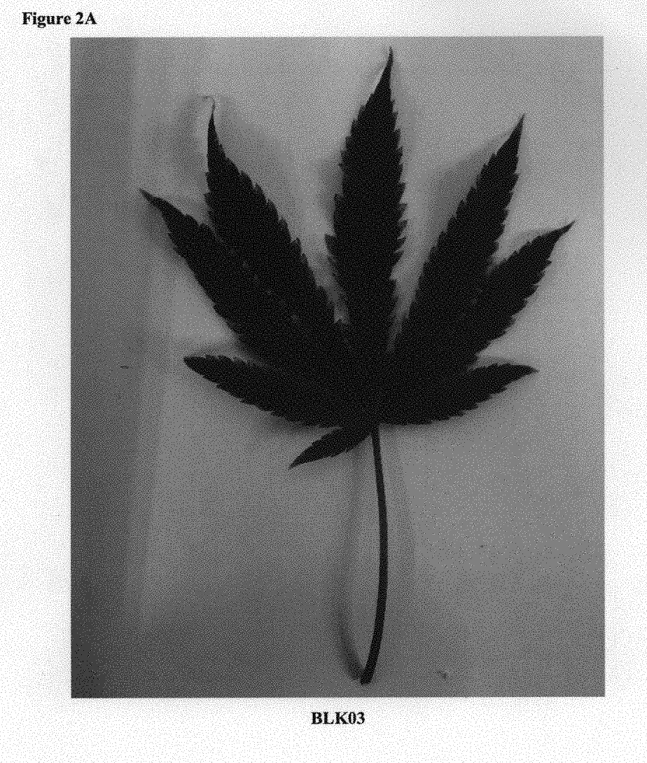

FIG. 2B shows a close view of a single leaf of the new variety ‘PRIMO CHERRY’ plant.

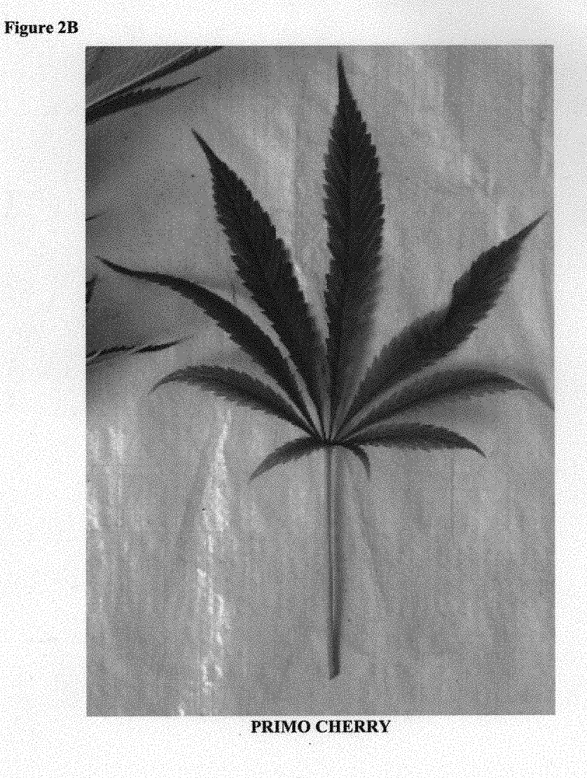

FIG. 3A shows top parts (including inflorescence) of the BLK03 plant from the side.

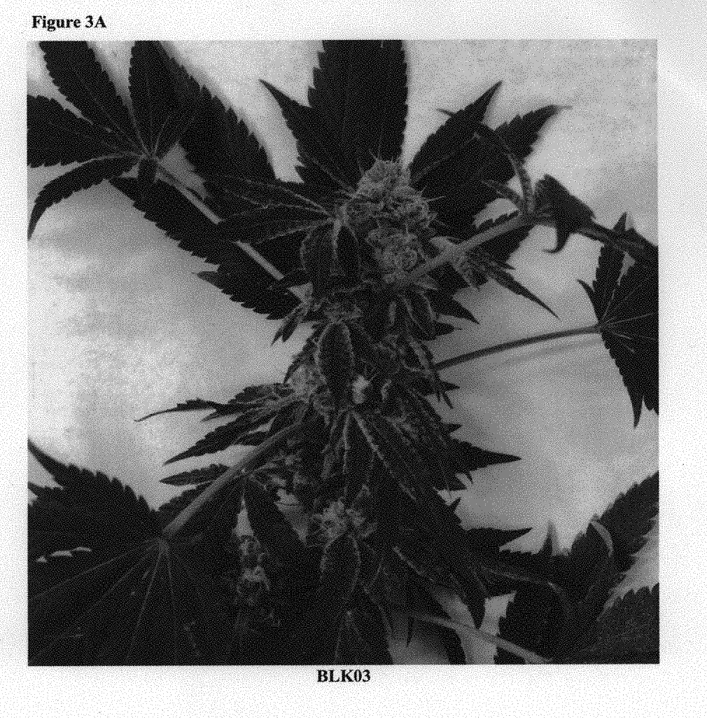

FIG. 3B shows top parts (including inflorescence) of the ‘PRIMO CHERRY’ plant from the side.

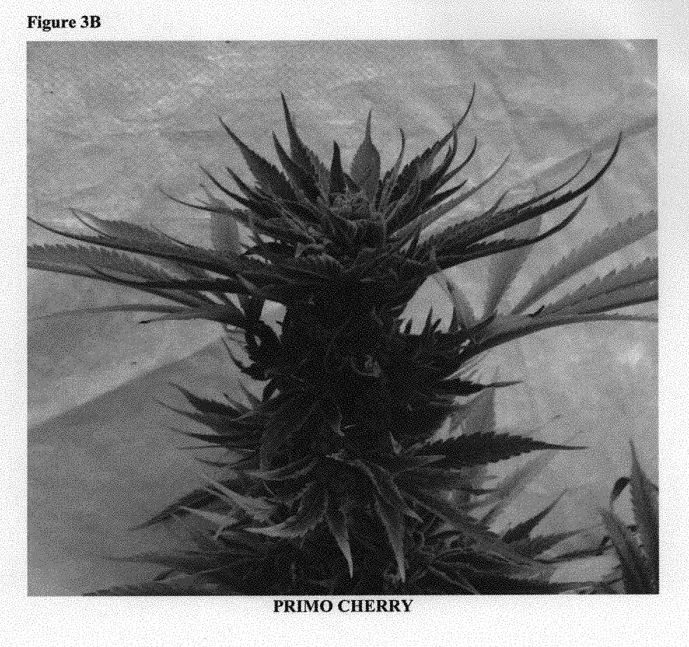

FIG. 4 shows a close view of flowers of the ‘PRIMO CHERRY’ plant at the late flowering/mature stage.

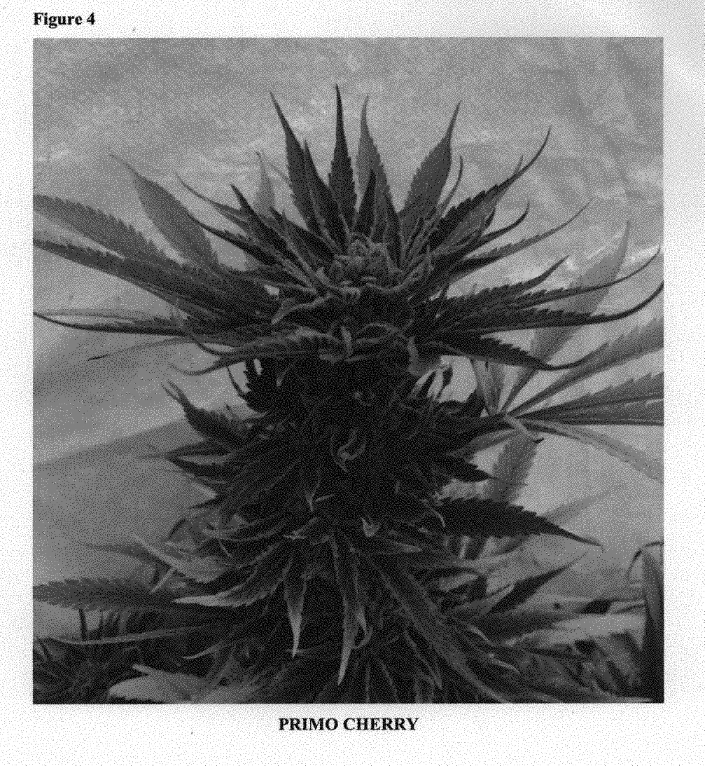

FIG. 5 shows another close view of flowers of the ‘PRIMO CHERRY’ plant at the late flowering/mature stage.

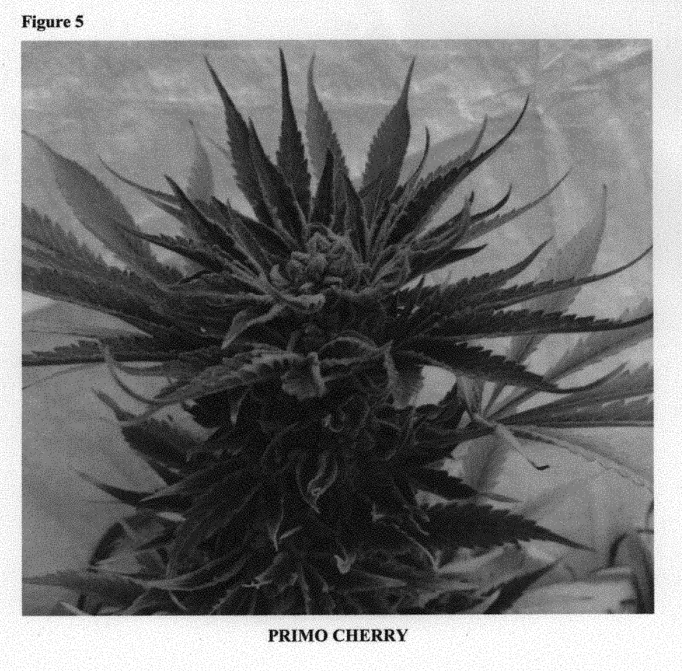

### DETAILED BOTANICAL DESCRIPTION

‘PRIMO CHERRY’ has not been observed under all possible environmental conditions, and the phenotype may vary significantly with variations in environment. The following observations, measurements, and comparisons describe this plant as grown at Salinas, Calif., when grown in the greenhouse, nursery or field, unless otherwise noted.

Plants for the botanical measurements in the present application are annual plants. In the following description, the color determination is in accordance with The Royal Horticultural Society Colour Chart, 1995 Edition and/or newer Editions such as 2001, 2007 and 2015, except where general color terms of ordinary dictionary significance are used.

The cannabis plant disclosed herein was derived from female and male parents that are internally designated as below.

A GNBR internal Code of the cannabis plant named ‘Primo Cherry’ is LC.SF.03. The variety name of ‘Primo Cherry’ is WP01.08.10.B4.P26.36.03. ‘Primo Cherry’ is a fertile hybrid derived from a controlled-cross between two proprietary cultivars WP01.P08.10 (WP01.P08.10; pollen accepter; female parent) and B4.Y3P26.36 (B4.Y3P26.36; pollen donor; male parent). A GNBR Breeding Code is (P08WP0115.10xP38BX08.10)x(B04xY03P26.36).03. The initial cross between two parental cultivars was made in October 2016. The primary phenotypic criteria used to select the new and distinct cannabis cultivar disclosed herein is as follows: structure score, nose/organoleptic testing, mold susceptibility/resistance, and insect susceptibility/resistance. Also, the first asexual propagation of ‘PRIMO CHERRY’ occurred on Jul. 20, 2017 in Salinas, Calif.

The following traits in combination further distinguish the cannabis cultivar ‘PRIMO CHERRY’ from the check variety, which is BLK03. ‘PRIMO CHERRY’ has been compared to, and can be distinguished from the check variety, ‘BLK03’ as follows. All plants were raised together and evaluated when 100 days old (i.e., 25 days in vegetative stage, 15 days in propagation stage, and 60 days in flowering times).

### I. GENERAL

| Characteristics | New Variety | Check Variety (BLK03) |
|---|---|---|
| Plant life forms | An herbaceous | An herbaceous |
| | plant (herb) | plant (herb) |
| Plant growth | An upright, | An upright, |
| habit | tap-rooted annual | tap-rooted annual |
| | plant; forming | plant; forming |
| | fibrous roots | fibrous roots |
| | when asexually | when asexually |
| | propagated | propagated |
| Plant origin | WP01.P08.10 X | GLD13 X BSIA |
| | B4.Y3P26.36 | |
| Plant | Asexually | Asexually |
| propagation | propagated by | propagated by |
| | cuttings and cloning | cuttings and cloning |
| Propagation ease | Easy | Moderate |
| Height | 1.5-2.0 m | 0.5-2.5 m |
| Width | 184 cm | 119.5 cm |
| Plant vigor | High | Medium |
| Time to Harvest | 11 weeks | 8 weeks |
| (Seed to Harvest) | | |
| Resistance to | Resistant | Resistant |
| pests or diseases | | |
| Genetically- | NO | NO |
| modified | | |
| organism | | |

### II. LEAF/FOLIAGE

| Characteristics | New Variety | Check Variety (BLK03) |
|---|---|---|
| Leaf | Alternate | Alternate |
| arrangement | | |
| Leaf shape | Palmately compound | Palmately compound |
| Leaf structure | Linear-lanceolate | Linear-lanceolate |
| | leaflet blades | leaflet blades |
| | with glandular hairs | with glandular hairs |
| Leaf margins | Dentate, coarsely | Dentate, coarsely |
| | serrated, and | serrated, and |
| | the teeth point | the teeth point |
| | away from the tip | away from the tip |
| Leaf hairs | Present | Present |
| Leaf length with | 36.0 cm | 16.6 cm |
| petiole at | | |
| maturity | | |
| Petiole length at | 13.1 cm | 6.5 cm |
| maturity | | |
| Petiole color | 144D | 140C |
| (RHS No.) | | |
| Stipule length at | 0.5 cm | 0.7 cm |
| maturity | | |
| Stipule shape | Scale-like-elliptical | Elliptical |
| Stipule color | 134B | 149B |
| (RHS No.) | | |
| No. of leaflets | 5-7 | 5-7 |
| Middle largest | 22.8 cm | 9.8 cm |
| (longest) leaflet | | |
| length | | |
| Middle largest | 4.7 cm | 2.3 cm |
| (longest) leaflet | | |
| width | | |
| Middle largest | 22.8:4.7 | 9.8:2.3 |
| (longest) leaflet | | |
| length/width | | |
| ratio | | |
| No. teeth of | 31 | 25 |
| middle leaflet | | |
| (average) | | |
| Leaf (upper | 140B | 132A |
| side) color | | |
| (RHS No.) | | |
| Leaf (lower | 141D | 134D |
| side) color | | |
| (RHS No.) | | |
| Leaf glossiness | Strong | Strong |
| Vein/midrib | Obliquely continuous | Obliquely continuous |
| shape | throughout leaflet | throughout leaflet |
| Vein/midrib | 150C | 144C |
| color (RHS No.) | | |
| Aroma | Cherry | Spicy |

### III. STEM

| Characteristics | New Variety | Check Variety (BLK03) |
|---|---|---|
| Stem shape | Hollow and ribbed | Hollow, ribbed, textured |
| Stem diameter | 4.5 cm | 2.8 cm |
| at base | | |
| Stem color | 139C | N144D |
| (RHS No.) | | |

IV. Inflorescence (Female/Pistillate Flowers)

| Characteristics | New Variety | Check Variety (BLK03) |
|---|---|---|
| Flowering | Elongated compound spikes or | Elongated compound spikes or |
| (blooming) | panicles, from 0.5 m-2.3 m in | panicles, from 0.5 m-1.2 m in |
| habit | length | length |
| Proportion of | 100% | 100% |
| female plants | | |
| Inflorescence | Above | Even |
| position | | |
| Flower | Overlapping | Overlapping-Touching |
| arrangement | | |
| Number of | 150-200 flowering sites | 350-550 flowering sites |
| flowers per | | |
| plant | | |
| Flower shape | More or less sessile and are borne | More or less sessile and are borne |
| | in racemes; calcarate-urceolate; a | in racemes; calcarate-urceolate; a |
| | small green bract enclosing the | small green bract enclosing the |
| | ovary with two long, slender | ovary with two long, slender |
| | stigmas projecting well above the | stigmas projecting well above the |
| | bract | bract |
| Flower | 0.7 cm | 0.7 cm |
| (individual | | |
| pistillate) length | | |
| Flower | 7.0 cm | 3.8 cm |
| (compound | | |
| cyme) diameter | | |
| Corolla | The inner envelope of floral | The inner envelope of floral |
| | leaves of a flower, of delicate | leaves of a flower, of delicate |
| | texture and of some color other | texture and of some color other |
| | than green | than green |
| Corolla Color | N/A | N/A |
| (RHS No.) | | |
| Bract shape | Urceolate | Urceolate |
| Bract color | 134B | N134C |
| (RHS No.) | | |
| Calyx shape | No defined calyx | No defined calyx |
| Calyx color | N/A | N/A |
| (RHS No.) | | |
| Stigma shape | Linear-pointed | Acute |
| Stigma length | 1.1 mm | 2.2 mm |
| Stigma color | 28C | 159D |
| (RHS No.) | | |
| Trichome shape | Capitate-stalked glandular | Capitate-stalked glandular |
| Trichome color | 157A | 157A |
| (RHS No.) | | |
| Terminal bud | Oblong | Oblong |
| shape | | |
| Terminal bud | 134B | 203C |
| color (RHS | | |
| No.) | | |
| Pedicel | Absent | Absent |
| Staminate shape | N/A | N/A |
| Pollen | Absent | Absent |
| description | | |
| Seed description | 1.5-2.3 mm; striped, smooth and | 1.8-2.3 mm, smooth and globular |
| | globular | |
| Petal | Apetalous | Apetalous |
| description | | |
| Max THC | About 12.38-15.24% | About 18.88-19.37% |
| content | | |
| Max CBD | About 5.40-7.13% | 0.00% |
| content | | |
| Max CBG | About 0.22-0.33% | About 0.84-0.91% |
| content | | |

### V. OTHER CHARACTERISTICS

| Characteristics | New Variety | Check Variety (BLK03) |
|---|---|---|
| Time period and | 7-9 weeks | 7-9 weeks |
| condition of | | |
| flowering/ | | |
| blooming | | |
| Hardiness of | Hardy to 25° F.-ambient | Hardy to 25° F.-ambient |
| plant | temperature | temperature |
| Breaking action | Flexible, highly resistant to | Strong, non-flexible |
| | breakage | |
| Rooting rate | 99%-vigorous | 70%-moderate |
| after | | |
| cutting/cloning | | |

The following is a detailed description of the new cultivar of ‘PRIMO CHERRY’.

The following description is for plants that are 100 days old as of the time of the measurements.
General description:
   Plant life form and habit.—An herbaceous, upright, tap-rooted annual plant, which forms fibrous roots when asexually propagated.
Classification:
   Denomination.—‘PRIMO CHERRY’.
   Species.—Cannabis hybrid (mixed background).
Origin, form, and growth characteristics;
   Origin.—Progeny of the controlled cross between WP01.P08.10 and B4.Y3P26.36.
   Propagation.—The strain is asexually propagated by cutting and cloning.
   Propagation ease.—Easy.
Plant:
   Height.—1.5-2.0 m.
   Width.—184 cm.
   Vigor.—High.
   Pest susceptibility.—Resistant.
   Time to harvest.—11 weeks.
   Genetically modified organism.—No.
Leaf/foliage:
   Structure.—Linear-lanceolate leaflet blades with glandular hairs.
   Shape.—Palmately compound.
   Arrangement.—Alternate.
   Margin.—Dentate, coarsely serrated, and the teeth point away from the tip.
   Hair.—Present.
   Leaf (with petiole) length at maturity.—36.0 cm.
   Number of leaflets.—5-7.
   Middle largest leaflet length.—22.8 cm.
   Middle largest leaflet width.—4.7 cm.
   Middle largest leaflet length/width ration.—22.8:4.7.
   Number of teeth of middle leaflet (average).—31.
   Color.—Upper side — 140B.
   Color.—Lower side — 141D.
   Leaf glossiness.—Strong.
   Veins/midrib shape.—Obliquely continuous throughout leaflet.
   Vein/midrib color.—150C.
Petiole:
   Petiole length.—13.1 cm.
   Petiole Color.—144D.
   Stipule shape.—Scale-like-elliptical.
   Stipule length.—0.5 cm.
   Stipule color.—134B.
Stem:
   Shape.—Hollow and ribbed.
   Diameter.—4.5 cm at base.
   Color.—139C.
Inflorescence:
   Blooming/flowering habit.—Elongated compound spikes or panicles, from 0.5-2.3 m in length.
   Inflorescence position relative to foliage.—Above.
   Flower arrangement.—Overlapping.
   Number of flowers per plant.—150-200 flowering sites.
Flowers:
   Shape.—More or less sessile and are borne in racemes; calcarate-urceolate; a small green bract enclosing the ovary with two long, slender stigmas projecting well above the bract.
   Flower (individual pistillate) length.—0.7 cm.
   Flower (compound cyme) diameter.—7.0 cm.
   Corolla shape.—The inner envelope of floral leaves of a flower, having delicate texture and some color other than green.
   Corolla color.—N/A.
   Bract shape.—Urceolate.
   Bract color.—134B.
   Stigma shape.—Linear-pointed.
   Stigma length.—1.1 mm.
   Stigma color.—28C.
   Trichome shape.—Capitate-stalked glandular.
   Trichome color.—157A.
   Cola (terminal bud).—Oblong.
   Cola (terminal bud)color.—134B.
   Pedicel.—Absent.
   Pedicel color.—N/A.
   Staminate flower.—N/A.
   Pollen.—Absent.
   Seed shape.—Striped, smooth and globular.
   Seed length.—1.5-2.3 mm.
   Petal.—Apetalous.
Other characteristics:
   Aroma.—Cherry.
   Flowering/blooming period.—7-9 weeks.
   Hardiness.—Hardy to 25° F.-ambient temperature.
   Breaking action.—Flexible, highly resistant to breakage.
   Rooting rate after cutting/cloning.—99% vigorous.

1. A new and distinct cultivar of cannabis plant named ‘PRIMO CHERRY’ substantially as shown and described herein.
